(19) United States
(12) Patent Application Publication
(10) Pub. No.: US 2010/0029986 A1
(43) Pub. Date: Feb. 4, 2010

(54) Novel amine functionalized carbon nanotube

(76) Inventor: David Abecassis, Huntington, NY (US)

Correspondence Address: Thomas A. O'Rourke;Bodner & O'Rourke, Suite 108, 425 Broadhollow Road, Melville, NY, 11747, US

(21) Appl. No.: 12/288,450

(22) Filed: Oct. 20, 2008

Related U.S. Application Data
(60) Provisional application No. 60/999,786, filed on Oct. 19, 2007.

(51) Int. Cl. C07C 211/43

(52) U.S. Cl. 564/308; 977/748; 977/750; 977/752; 977/847

(57) ABSTRACT

Methods of forming a multi walled or single walled carbon nanotube with one or more amine groups on the surface thereof are described. The method includes reacting a carbon nanotube having a hydroxyl surface group or a carboxyl surface group with ammonia in the presence of a catalyst at a temperature of about 300° C. or more.

This application claims priority on U.S. Application Ser. No. 60/999,786 filed Oct. 19, 2007, the disclosures of which are incorporated herein by reference.

### FIELD OF THE INVENTION

The field of the invention is nanoparticle synthesis for carbon nanotube surface functionalization. More particularly, the field of the invention is nanotubes with amine functional groups on the surface of the nanotube.

### BACKGROUND OF THE INVENTION

Carbon nanotubes are an allotrope of carbon. Allotropes are different forms of the same element. Carbon has several allotropes. Elemental carbon can be found in several forms including amorphous graphite, and diamonds. In diamonds, the carbon atoms are bonded together in a tetrahedral lattice arrangement. For graphite, the carbon atoms are bonded together in sheets of a hexagonal lattice. Carbon nanotubes are a relatively new type of allotrope whose uses are just now being explored. Carbon nanotubes are cylindrical carbon molecules. A nanotube is a member of the fullerene structural family. The diameter of a nanotube is usually just a few nanometers in size. Nanotubes can be up to about several centimeters in length. There are two main varieties of nanotubes, single walled nanotubes and multiwalled nanotubes.

Carbon nanotubes are the result of sheet-like structures called graphenes which are one or a few carbon atoms thick. In particular, carbon nanotubes have superconductive electrical and thermal properties when their crystalline structure is formed by the rolling up of one sheet of carbon atoms (graphene) into a tube. Carbon nanotubes can be both electrically semi, and super conductive materials. Because of this conductivity, they are being explored for use in both biological and material systems. The value of the amine functionalization is that it reacts and forms chemical bonds with other functional organic groups such as carboxylic acid groups (COOH) and aldehyde (CHO) carrying organic and biomolecules. This allows carbon nanotubes to be incorporated into other systems such as synthetic and biological polymers at the molecular level.

### OBJECTS OF THE INVENTION

One object of the invention is to produce a new type of carbon nanotube.

Yet another object is to produce an amino-functionalized carbon nanotube.

Still another object of the invention is to produce an amine synthesized carbon nanotube for specific solubility and synthesis.

It is a still further object of the invention to provide an amine synthesized carbon nanotube for use as an additive into other materials.

It is also an object of the invention to provide an amine synthesized carbon nanotube where the amine is on the outer surface of the cylindrical nanotube.

### SUMMARY OF THE INVENTION

The invention relates to the formation of hydroxyl functionalized or acid functionalized carbon nanotubes (CNT-OH, CNT-COOH) that are reacted with an amino based compound to arrive at an amine functionalized carbon nanotube (CNT-NH2) via chemical synthesis. The carbon nanotubes of the present invention preferably have an outer diameter of less than about 100 nm. For single walled carbon nanotubes the preferred outer diameter is less than about 2 nm. Although the invention is primarily directed to carbon nanotubes, the invention also has applicability to Fullerenes & Carbon Nanofibers. In another embodiment, the outer diameter of the carbon nanotubes, Fullerenes, or Carbon Nanofibers of the present invention preferably have a diameter of less than about 150 nm.

### DETAILED DESCRIPTION OF THE INVENTION

Carboxylic acid functionalized single walled carbon nanotube can be prepared by a variety of reactions. One reaction entails the reaction of a carbon nanotube with a mixture of concentrated sulphuric and nitric acid. Sonification is one way of performing this reaction. Multi-walled carbon nanotube functionalized with carboxylic acid can also be prepared in a similar manner. Carboxylic functionalized carbon nanotubes are also available from Cheap Tubes, Inc. These particular nanotubes are produced with nitric acid. Carboxylic acid functionalized carbon nanotubes can also be formed by reactions with potassium permanganate and by reaction with sulfuric acid and hydrogen peroxide. Hydroxy functionalized carbon nanotubes can be formed, for example, by reacting fluoronanotubes with a series of diols and glycols in the presence of alkali. Other reactions are available for adding hydroxyl functionality to the carbon nanotube.

A carbon nanotube which has hydroxyl or carboxylic functional groups on a surface such as an outer surface is placed in a reaction vessel with the appropriate catalyst and an excess of ammonia gas. The mixture is heated to a temperature of about 300° C. or more in the presence of the catalyst and yields primary amine groups where a hydroxyl or carboxylic groups are located on the precursor molecule.

Suitable catalysts include aluminum (Al₂O₃), a zinc salt, a cupric salt, sulfuric acid, copper (I) oxide or a clay.

In a first embodiment, the hydroxyl group(s) on the carbon nanotubes reacts with the ammonia gas (NH₃) in the presence of Al₂O₃ at about 300° C. to form a carbon nanotube with amine functionality (C—NH_{z}). The nanotube can be a single walled nanotube or a multi-walled nanotube.

In another embodiment, a single or multi-walled carbon nanotube is chlorinated with hydrochloric acid in the presence of zinc chloride and a desiccant to provide a carbon nanotube with a halogen intermediate. This intermediate has one or more chlorine groups on the surface of the carbon nanotube. The halogen intermediate is converted to a primary amine in a second step. The halogen intermediate is reacted with HCl in a Hoffman reaction by ammonia substitution of the halogen function.

In an alternate embodiment, a single or multi-walled carbon nanotube with one or more surface carboxyl groups (COOH) is thermally decarboxylated in the presence of a mineral catalyst and ammonia gas to form primary amine (NH_{z}) functionality on the surface of the carbon nanotube. The mineral catalyst can include magnesium oxide, aluminum oxide, cupric sulfate, ion pyrite, copper (I) oxide.

Other catalysts can include concentrated sulfuric acid or a clay such as a montmorellonite clay.

### EXAMPLES OF THE INVENTION

### Example 1

Single walled nanotubes of an outer walled diameter of 1-2 nanometers which have a carboxyl group on the surface of the carbon nanotube are placed in a reaction vessel in an atmosphere of ammonia with and brought to the temperature of 350 degrees Celsius in the presence of Zinc Chloride and using sodium sulfate as a catalyst. The product is removed after four hours and now contains amine groups as revealed by IR absorption at 1600 cm-1.

### Example 2

Multi walled hydroxylated carbon nanotubes of 50 nm diameter are added to a reaction vessel with ammonia gas and alumina (Al₂O₃) powder and brought to 300 degrees Celsius for a period of two hours under sealed reactor conditions. The carbon nanotubes are measured for groups using IR and reveal increased absorption at 1600 cm-1(NH) and a decrease in absorption peaks at 3500cm-1(OH).

What is claimed:

1. A method of forming a multi walled or single walled carbon nanotube with one or more amine groups on the surface thereof comprising reacting a carbon nanotube having a hydroxyl surface group or a carboxyl surface group with ammonia in the presence of a catalyst at a temperature of about 300° C. or more.

2. The method according to claim 1 wherein the hydroxyl group or carboxyl group is amine substituted in the presence of ammonia gas and Al₂O₃ at 300° C.

3. A method of forming a multi walled or single walled carbon nanotube with one or more amine groups on a surface of the carbon nanotube comprising forming a carbon nanotube with a hydroxyl surface group, chlorinating the carbon nanotube with HCl (hydrochloric acid) in the presence of a catalyst and a desiccant to produce a halogen intermediate, converting the chlorinated intermediate to a primary amine in a second step in the presence of HCl(hydrochloric acid) in a Hoffman reaction by ammonia substitution of the halogen function.

4. The method according to claim 1 wherein the catalyst is zinc chloride.

5. The method according to claim 1 wherein the catalyst is sulfuric acid.

6. A method of forming a multi walled or single walled carbon nanotube with one or more amine groups on a surface of the carbon nanotube the method comprising reacting a carbon nanotube having an organic acid (COOH) surface group with NH₃ gas in the presence of a catalyst to form a primary amine on the surface of the carbon nanotube.

7. The method according to claim 6 where the catalyst is concentrated sulfuric acid.

8. The method according to claim 6 where the catalyst is magnesium oxide

9. The method according to claim 6 where the catalyst is aluminum oxide.

10. The method according to claim 6 where the catalyst is cupric sulfate.

11. The method according to claim 6 where the catalyst is montmorillonite clay.

12. The method according to claim 6 where the catalyst is iron pyrite.

13. The method according to claim 6 where the catalyst is a Copper(I) oxide.

14. A carbon nanotube having one or more NH₂ groups on the surface thereof, said NH₂ groups being formed be the reaction of a canbon nanotube having carboxyl or hydroxyl functionality on a surface of the nanotube with ammonia gas in the presence of a catalyst at a temperature of about 300 degrees C. or more.
